(19) United States
(12) Patent Application Publication
(10) Pub. No.: US 2009/0308730 A1
(43) Pub. Date: Dec. 17, 2009

(54) Hollow cathode plasma source for bio and chemical decotaminiation of air and surfaces

(76) Inventors: S. Ismat Shah, Wilmington, DE (US); Anshu Pradhan, Fremont, CA (US)

Correspondence Address: RATNERPRESTIA, P.O. BOX 1596, WILMINGTON, DE, 19899, US

(21) Appl. No.: 11/989,562

(22) PCT Filed: Jul. 28, 2006
(86) PCT No.: PCT/US2006/029221

§ 371 Date: May 22, 2009

Related U.S. Application Data
(60) Provisional application No. 60/703,417, filed on Jul. 29, 2005.

(51) Int. Cl. B01J 19/08; B01D 53/34; A61L 2/28

(52) U.S. Cl. 204/164; 422/183; 422/119

(57) ABSTRACT

Hollow cathode source pollution abatement systems for the remediation of chemical and microbiological pollutants are disclosed. The systems comprise a plasma generator having a hollow cathode source (HCS) for generating plasma to break down pollutant chemicals and microorganisms into simpler byproducts. The byproducts are trapped on an inner surface of the HCS. The systems allow for elimination of pollutant chemicals and microorganisms from air or gas streams as well as from the surfaces of articles. Methods of operating such systems are also disclosed.

### FIELD OF THE INVENTION

The disclosure relates to the abatement of airborne chemical or biological pollutants.

### BACKGROUND OF THE INVENTION

It is known to use non-thermal plasma devices for elimination of chemical and biological agents. For example, Prospects for Non-thermal Atmospheric Plasmas for Pollution Abatement provides an overview of different non-thermal plasma technologies. See, Prospects for Non-thermal Atmospheric Plasmas for Pollution Abatement, R. McAdams, AEA Technology, Cutham Science Centre, Abingdon, Oxfordshire 0X14 3ED, UK 3. Phys. D: Appl. Phys. 34 (2001), pp. 2810-2821. In non-thermal plasmas, the electrons have a significantly higher temperature compared to the ions, atoms and molecules. The McAdams paper provides an overview of both the technologies involved and the diverse potential application areas. A general description of these atmospheric plasmas and the basic principles involved in the destruction or removal of gaseous phase pollutants, based on the nature of the processes taking place within these plasmas, is given. Several examples of the different plasma technologies are described. The technologies described include pulsed corona, microwave and dielectric barrier plasma technologies. Their suitability and use in various application areas are also discussed, including incinerator off-gas treatment, industrial process off-gas treatment and diesel exhaust after-treatment.

H. W. Herrmann, I. Henins, J. Park and G. S. Selwyn have disclosed the use of an atmospheric pressure plasma jet for neutralizing chemical warfare agents. According to Herrmann et al., the atmospheric pressure plasma jet (APPJ) is a nonthermal, high pressure, uniform glow plasma discharge that produces a high velocity effluent stream of highly reactive chemical species. The discharge operates on a feedstock gas (e.g., He/O₂/H₂O!), which flows between an outer, grounded, cylindrical electrode and an inner, coaxial electrode powered at 13.56 MHz rf. While passing through the plasma, the feedstock gas becomes excited, dissociated or ionized by electron impact. Once the gas exits the discharge volume, ions and electrons are rapidly lost by recombination, but the fast-flowing effluent still contains neutral metastable species (e.g., O₂, He*) and radicals (e.g., O, OH). This reactive effluent has been shown to be an effective neutralizer of surrogates for anthrax spores and mustard blister agent. See, Decontamination of Chemical and Biological Warfare (CBW) Agents Using an Atmospheric Pressure Plasma Jet (APPJ), H. W. Herrmann, I. Henins, J. Park, and G. S. Selwyn, Physics Division, Los Alamos National Laboratory, Los Alamos, N. Mex. 87545, Physics of Plasmas, 6, 2284 (1999).

Additionally, a process using microwave plasmas for the destruction of perfluorocompounds has been experimentally investigated. The plasma device for this process was operated at atmospheric pressure and at a frequency of 2.45 GHz in synthetic gas mixtures containing N₂ and CF₄. It was found that the perfluorocompound destruction and removal efficiency of the process is highly dependent on the total gas flow and concentration of CF₄. Destruction and removal efficiencies of CF₄ up to 98% have been achieved using 1.9 kW of microwave power at 16 L/min total flow rate. See, Studies of 2.45 GHz Microwave Induced Plasma Abatement of CF4, Marilena Radoui, BOC Edwards, Exhaust Gas Management, Kenn Business Park, Kenn Road, BS21 6TH Clevedon, U.K., Environ. Sci. Technol. 2003, 37, 3985-3988.

Conventional plasma-based chemical and biological agent abatement devices, such as those disclosed in the literature discussed above, are relatively complex. Additionally, such devices often require high voltage power generators. It is therefore desirable to provide devices and methods for eliminating chemical and biological pollutants that are less complicated and less expensive than known plasma-based pollution abatement devices and methods. It is further desirable to provide a devices and processes for the elimination of chemical and biological agents that require lower power consumption than known devices and methods.

### BRIEF SUMMARY OF THE INVENTION

The disclosure concerns systems and methods for eliminating chemical and biological pollutants. The systems and methods employ a hollow cathode plasma generator that breaks down chemicals and microorganisms into byproduct compositions and traps the byproduct compositions inside the plasma generator.

According to one embodiment, a pollution abatement system comprises:

a plasma generator, wherein said plasma generator comprises a hollow cathode source disposed in a housing and an anode spaced from said hollow cathode source;

a power supply connected to said plasma generator;

a feed gas supply comprising a feed gas suitable for generating plasma within an inner volume of the hollow cathode;

a feed gas tube extending from said feed gas supply into the inner volume of said hollow cathode source and arranged to carry a first gas stream comprising the feed gas;

a contaminated gas source comprising a contaminated gas;

a contaminated gas tube in communication with said contaminated gas source and connected to introduce a second gas stream comprising the contaminated gas to said first gas stream; and

a gas outlet in communication with an inner chamber of the housing and arranged to remove remediated gas from said plasma generator.

The feed gas tube and the contaminated gas tube may be separate gas tubes in selective communication with each other, wherein the contaminated gas is introduced from the contaminated gas tube into the feed gas tube as a second gas stream. Alternatively, the feed gas tube and the contaminated gas tube may be the same gas tube, wherein the feed gas includes the contaminated gas.

According to another embodiment, a pollution abatement system comprises:

a plasma generator, wherein said plasma generator comprises a hollow cathode source disposed in a housing and an anode spaced from said hollow cathode source;

a power supply connected to said plasma generator;

a feed gas supply comprising a feed gas suitable for generating plasma within an inner volume of the hollow cathode;

a feed gas tube extending from said feed gas supply into the inner volume of said hollow cathode source and arranged to carry a first gas stream comprising the feed gas;

a gas outlet in communication with an inner chamber of the housing and arranged to remove gas from said plasma generator; and

a contaminated article disposed inside the hollow cathode source and arranged to be decontaminated by exposure to the plasma, wherein the contaminated article is contaminated by at least one of the following agents: a chemical pollutant, a volatile organic compound and a microorganism.

Further embodiments are related to methods for decontaminating gases and surfaces. According to one embodiment, a method for decontaminating a gas comprises:

providing a plasma generator, wherein said plasma generator comprises a hollow cathode source disposed in a housing and an anode spaced from said hollow cathode source;

powering said plasma generator with a power source;

supplying a feed gas to an inner volume of the hollow cathode so as to generate plasma within said inner volume;

supplying a contaminated gas to said inner volume and passing said contaminated gas through said plasma so as to break down said contaminated gas into remediated gas and byproducts; and

removing said remediated gas from said plasma generator.

According to another embodiment, a method for decontaminating a gas comprises:

providing a plasma generator, wherein said plasma generator comprises a hollow cathode source disposed in a housing and an anode spaced from said hollow cathode source;

powering said plasma generator with a power source;

supplying a feed gas to an inner volume of the hollow cathode so as to generate plasma within said inner volume, wherein said feed gas includes at least one contaminant;

passing said at least one contaminant through said plasma so as to break down said at least one contaminant into byproducts, and thereby remediating said feed gas; and

removing remediated gas from said plasma generator.

According to yet another embodiment, a method for decontaminating an article comprises:

providing a plasma generator, wherein said plasma generator comprises a hollow cathode source disposed in a housing and an anode spaced from said hollow cathode source;

placing the article inside the hollow cathode source, wherein a surface of the article is contaminated by at least one of the following agents: a chemical pollutant, a volatile organic compound and a microorganism;

powering said plasma generator with a power source;

supplying a feed gas to an inner volume of the hollow cathode so as to generate plasma within said inner volume;

exposing said surface of said article to said plasma so as to break down said agent into byproducts; and

removing uncontaminated gas from said plasma generator.

Further features and advantages of the invention will become apparent from the following description and drawings.

### BRIEF DESCRIPTION OF THE DRAWINGS

FIG. 1 is a schematic illustration of a pollution abatement system according to one embodiment;

FIG. 2 is a schematic illustration of a pollution abatement system according to another embodiment;

FIG. 3 is a schematic illustration of a pollution abatement system for the decontaminating surfaces;

FIGS. 4-5 are schematic illustrations of experimental pollution abatement systems;

FIGS. 6A-6F are mass spectrometer chromatographs showing the remediation of benzene using the system of FIG. 4.

FIGS. 7A and 7B are multiple ion detection (MID) plots from a mass spectrometer demonstrating the remediation of benzene using the system of FIG. 4;

FIGS. 8A-8E are mass spectrometer chromatographs showing the remediation of benzene using the system of FIG. 5.

FIGS. 9A and 9B are MID plots from a mass spectrometer demonstrating the remediation of benzene using the system of FIG. 5; and

FIG. 10 is a comparison of the MID plots for the systems of FIGS. 4 and 5.

### DETAILED DESCRIPTION OF THE INVENTION

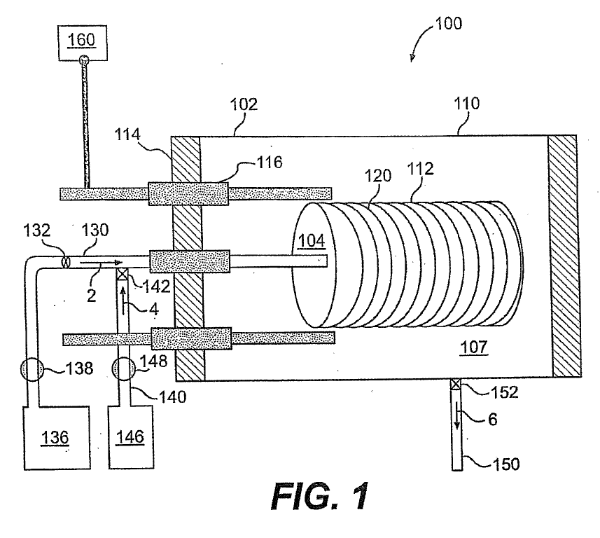

FIG. 1 shows a pollution abatement system 100 which includes a hollow cathode source plasma generator 110. The plasma generator 110 includes a hollow cathode source (HCS) 112 enclosed in a housing 102, and an anode 114 axially spaced and insulated from the HCS 112 by an insulator 116. The HCS 112 is connected to a power supply 160 and the anode 114 is electrically grounded. Cooling water tubes 120 are disposed around the HCS 112. The housing 102 electrically isolates the HCS 112 from the atmosphere to avoid plasma formation on the surface rather than the inside of the HCS 112.

A feed gas tube 130 extends from a feed gas supply 136 through the housing 102 and into an inner volume 104 of the HCS 112. The feed gas supply tube 130 carries a feed gas stream 2 which may comprise air or another gas suitable for generating plasma within the generator 110. The feed gas may be supplied through the feed gas supply tube 130 from the feed gas supply 136 by a pump 138 or other suitable device. The system further includes a contaminant source 146. The contaminant source 146 may contain one or more gases including pollutant chemical compounds, volatile organic compounds (VOCs) or microorganisms. A contaminant supply tube 140 connects the contaminant source 146 to the feed gas supply tube 130 and carries a contaminated gas stream 4 from the contaminant source 146. A pump or other delivery device 148 may be provided for feeding the contaminated gas stream 4 through the supply tube 140. The system 100 further includes a gas outlet tube 150 extending from the interior chamber 107 of the housing 102 to an area outside of the plasma generator 110. The gas outlet tube 150 allows harmless, remediated gas 6 to be removed from the inner chamber 107. The feed gas tube 130, contaminant supply tube 140 and gas outlet tube 150 may be provided with valves 132, 142, 152 and associated controls (not shown) in order to maintain a desired pressure within the cathode inner volume 104.

In the operation of the system 100, the plasma generator 110 is powered and feed gas stream 2 is fed through the feed gas tube 130 to the inner volume 104 of the HCS 112, causing the generation of plasma in the inner volume 104 in a known manner. As the feed gas stream 2 flows through the feed gas tube 130, the polluted gas stream 4 is introduced into the feed gas tube 130 through the supply tube 140. The contaminated gas stream 4 mixes with the feed gas stream 2 in the feed gas tube 130. Thereafter, the contaminated gas stream 4 is introduced inside the HCS 112 where it interacts with the plasma. The plasma generator can be operated with the inner volume 104 at pressures as low as 1 mTorr. Therefore, the pressure of the polluted gas stream 4 and the feed gas stream 2 can be as low as 1 mTorr.

As the polluted gas stream 4 passes through the plasma, the plasma breaks down pollutant chemical compounds, volatile organic compounds (VOCs) or microorganisms in the polluted gas stream 4. Chemical compounds, VOCs or microorganisms are broken down into simpler byproduct waste molecules such as CO, O₂, CO₂ and H₂O. The byproduct waste molecules are removed from the gas phase and trapped on the inner surface of the HCS 112. As plasma generation (i.e., the plasma “on” phase) continues and byproduct waste molecules collect on the inner surface of the HCS 112, the pressure of the inner volume 104 of the HCS 112 falls. Once the pressure of the inner volume 104 falls below a certain level (typically below about 1 mTorr), the plasma becomes unstable and plasma generation ceases (i.e., the plasma “off” phase). Once plasma generation ceases, the pressure of the inner volume 104 rises until it reaches a level at which plasma generation begins again.

The inventive system and method goes beyond the chemical cleaving provided by prior art pollution remediation devices. As described above, the inventive system and method provides complete elimination of pollutants and trapping of waste molecules inside the HCS 112. Waste can be removed from the plasma generator 110 by simply cleaning off the inner surface of the HCS 112. Additionally, in contrast to more complex, conventional pollution abatement systems which use microwave or corona discharge-type plasma generation and usually require high voltage power generators, the inventive system 100 can be operated with a relatively low voltage power supply 160.

The inventive system 100, and the individual components thereof, can be scaled for use in various applications. Such applications include, but are not limited to, residential and industrial air cleaning systems and industrial waste gas cleaning systems. Where the system 100 is an air cleaning system, the pollutant source 146 would be a room or building and the polluted gas stream 4 would comprise room or building air that is to be remediated by exposure to the plasma. Where the system 100 is an industrial exhaust gas processing system, the pollutant source 146 would be equipment used in an industrial process and the polluted gas stream 4 would comprise waste gas generated by the industrial process, wherein the waste gas is to be remediated prior to release into the atmosphere.

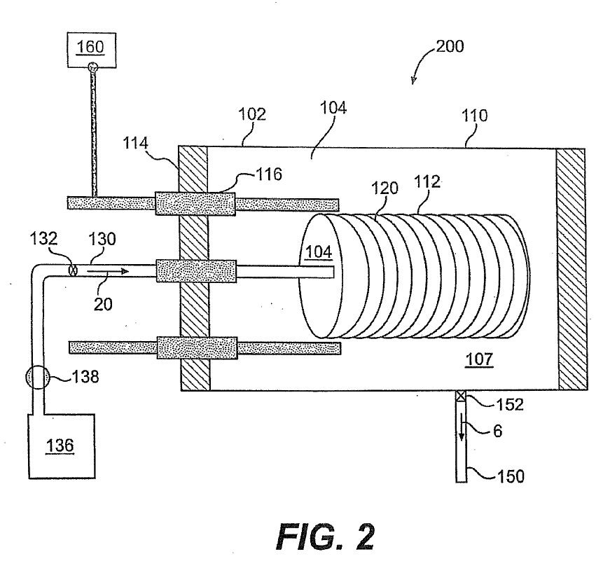

FIG. 2 shows a system 200 according to another embodiment, in which the feed gas supply 136 contains polluted gas. Therefore, there is no need for a separate pollutant source and supply tube. Referring to FIG. 2, in which reference numbers repeated from FIG. 1 indicate similar components, the system 200 includes a hollow cathode source plasma generator 110 including HCS 112 enclosed in a housing 102, and an anode 114 axially spaced and insulated from the HCS 112 by insulator 116. The HCS 112 is connected to power supply 160 and the anode 114 is electrically grounded. Cooling water tubes 120 are disposed around the HCS 112. The housing 102 electrically isolates the HCS 112 from the atmosphere to avoid plasma formation on the surface rather than the inside of the HCS 112.

A feed gas tube 130 extends from a feed gas supply 136 through the housing 102 and into the inner volume 104 of the HCS 112. The feed gas supply tube 130 carries a feed gas stream 20 which may comprise air or another gas suitable for generating plasma within the generator 110. The feed gas stream 20 further comprises one or more pollutants, which may include gaseous chemicals and/or microorganisms. The feed gas stream 20 may be supplied through the feed gas supply tube 130 from the feed gas supply 136 by a pump 138 or other suitable device. The system 200 further includes a gas outlet tube 150 extending from the interior volume 104 of the HCS 112 to an area outside of the plasma generator 110. The gas outlet tube 150 allows harmless, remediated gas 6 to be removed from the inner volume 104. The feed gas tube 130 and gas outlet tube 150 may be provided with valves 132, 152 and associated controls (not shown) for maintaining a desired pressure within the cathode inner volume 104.

In the operation of the system 200, the plasma generator 110 is powered and feed gas stream 20 is fed through the feed gas tube 130 to the inner volume 104 of the HCS 112, causing the generation of plasma in the cathode inner volume 104 in a known manner. As indicated with regard to the previous embodiment, the plasma generator can be operated with the inner volume 104 at pressures as low as 1 mTorr. Therefore, the pressure of the feed gas stream 20 can be as low as 1 mTorr.

As plasma is generated from the feed gas stream 20, the plasma breaks down pollutant chemical compounds, volatile organic compounds (VOCs) and/or microorganisms in the feed gas stream 20. As is the case in the previous embodiment, the chemical compounds, VOCs or toxic microorganisms are broken down into simpler byproduct waste molecules such as CO, O₂, CO₂ and H₂O, and the byproduct waste molecules are removed from the gas phase and trapped on the inner surface of the HCS 112. As plasma generation (i.e., the plasma “on” phase) continues and byproduct waste molecules collect on the inner surface of the HCS 112, the pressure of the inner volume 104 of the HCS 112 falls. Once the pressure of the inner volume 104 falls below a certain level (typically below about 1 mTorr), the plasma becomes unstable and plasma generation ceases (i.e., the plasma “off” phase). Once plasma generation ceases, the pressure of the inner volume 104 rises until it reaches a level at which plasma generation begins again.

Thus, the operation of systems 100 and 200 is similar, except that the system 200 does not include a separate pollutant source, since the pollutants originate from the feed gas supply 136.

As is the case the with system 100, the system 200 may be scaled for use in various applications, including residential and industrial air cleaning systems or industrial waste gas cleaning systems. In an air cleaning system, the feed gas supply 136 would be a room or building and the feed gas stream 20 would comprise polluted air in the room or building. Alternatively, in an industrial waste gas cleaning system, the feed gas supply 136 would be equipment used in an industrial process and the feed gas stream 20 would comprise polluted waste gas generated by the industrial process.

According to another embodiment, the system 200 may be an exhaust gas cleaning system for a motor vehicle. In such an embodiment, the plasma generator 110 may be a catalytic converter, in which case the feed gas supply 136 would be an engine and the feed gas stream 20 would comprise exhaust gases generated by the engine.

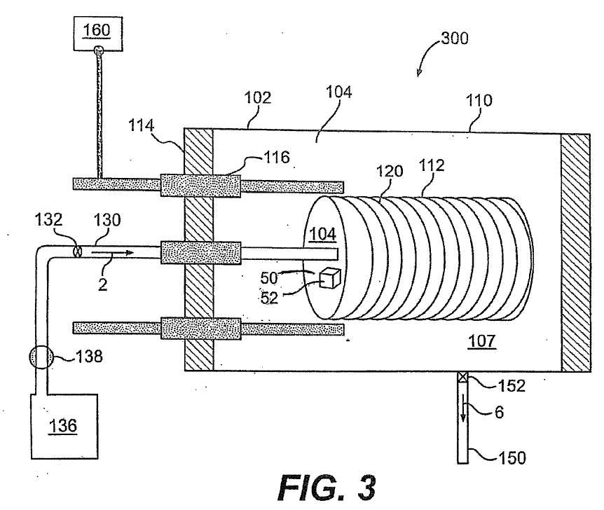

An embodiment of a pollution abatement system for cleaning surfaces is shown in FIG. 3. As shown in FIG. 3, system 300 is similar to system 200, except that it comprises a feed gas stream 2, which may comprise any suitable feed gas, and an article 50 having a surface 52 that is to be cleaned by the plasma generator 110. The surface 52 is contaminated by chemicals or microbiological organisms.

In operation of the system 300, the article 50 is first placed in the inner volume 104 of the HCS 112. Plasma is generated as described with respect to the embodiment of FIG. 2. As plasma is generated from the feed gas stream 2, the plasma breaks down chemical compounds, volatile organic compounds (VOCs) and/or microorganisms on the surface 52 of the article 50 into byproduct waste molecules such as CO, CO₂ and H₂O. The byproduct waste molecules are trapped on the inner surface of the HCS 112. Uncontaminated gas 6 is removed via the gas outlet tube 150. As plasma generation (i.e., the plasma “on” phase) continues and byproduct waste molecules collect on the inner surface of the HCS 112, the pressure of the inner volume 104 of the HCS 112 falls. Once the pressure of the inner volume 104 falls below a certain level (again, typically below about 1 mTorr), the plasma becomes unstable and plasma generation ceases (i.e., the plasma “off” phase). Once plasma generation ceases, the pressure of the inner volume 104 rises until it reaches a level at which plasma generation begins again. This process may be repeated until the article 50 is substantially entirely decontaminated.

The following examples discuss the implementation of certain experimental embodiments of a device and method for the elimination of chemical and biological agents.

### Example 1

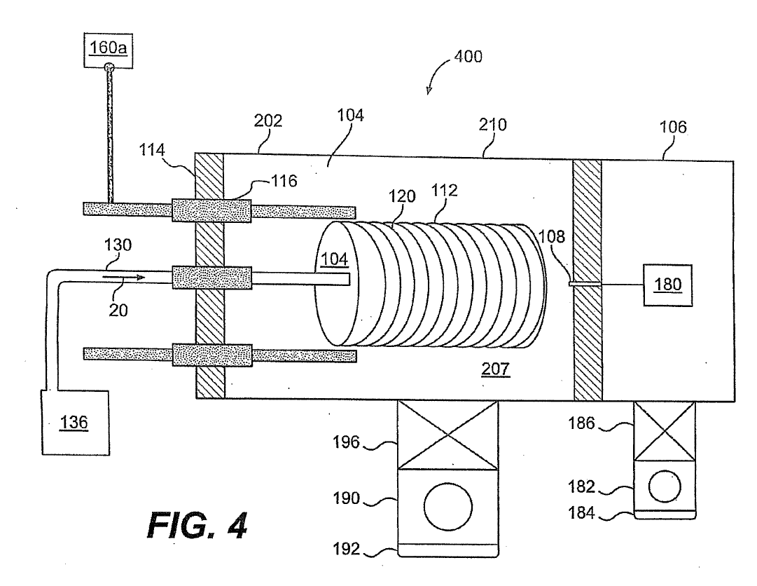

An experimental hollow cathode source pollution abatement system 400, illustrated in FIG. 4, was constructed. Reference numbers repeated from FIGS. 1-3 indicate similar components. The system 400 included a plasma generator 210 comprising a hollow HCS 112 disposed in the inner chamber 207 of a housing 202, and an anode 114 axially spaced and insulated from the HCS 112 by an insulator 116. The HCS, or target 112 was made of an aluminum tube having a length of 5 inches and a diameter of 2.5 inches. Copper cooling water tubes 120 were wrapped around HCS 112. The anode 114 was made from aluminum disks. The hollow cathode/cooling water tube assembly was insulated with Kapton tape (not shown). The device 210 was connected to a 15 watt power supply 160a. The housing 202 was constructed to electrically isolate the HCS 112 from the atmosphere to avoid plasma formation on the outer surface rather than the inside of the HCS 112. The housing 202 was further constructed to maintain a vacuum inside the device 210 in order to allow for the performance of mass spectrometry without the complication of residual gases in the device 210.

An aluminum feed gas tube 130 was provided at a first end of the system 400 for the introduction of feed gas stream 20 into the inner volume 104 of the hollow cathode. The feed gas tube 130 was arranged such that the gas inlet was placed one inch inside the HCS 112. A feed gas reservoir 136 was connected to the feed gas tube 130 through a needle valve (not shown). Benzene was selected as the feed gas for the system, and was filled into the feed gas reservoir 136.

The system 400 was further provided with a 120 L/s turbomolecular pump (TMP) 190 to create vacuum in the device 210 so that the benzene could be fed into the system. The TMP 190 was backed by a foreline pump 192, which was provided to take away the gas pumped out by the TMP 190. A gate valve 196 was provided to control the flow to the TMP 190. The base internal pressure of the system (i.e., the lowest pressure obtained before the start of the experiment) was 4×10⁻⁷ Torr.

A differentially pumped quantum mass spectrometer (QMS) 180 was provided in a chamber 106 at a second end of the system 400 to enable the detection of byproduct molecules generated by the breakdown of benzene in the generator 210. The chamber 106 for the QMS 180 was connected to the inner volume 104 through a 10 μm orifice 108 and was connected to a TMP 182 backed by a foreline pump 184. The TMP 182 was provided to supply molecules to the QMS 180 for mass spectrometry. The foreline pump 184 was provided to take away the gas pumped out by the TMP 182. A gate valve 186 was provided to control the flow to the TMP 182.

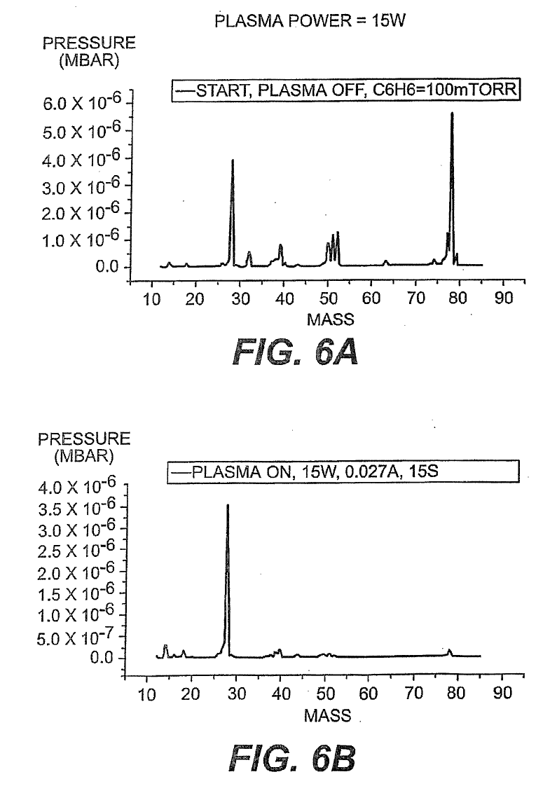

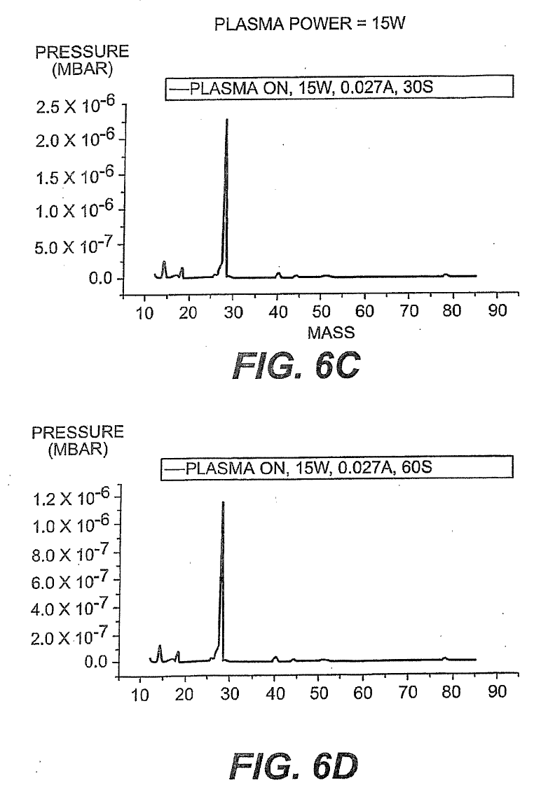

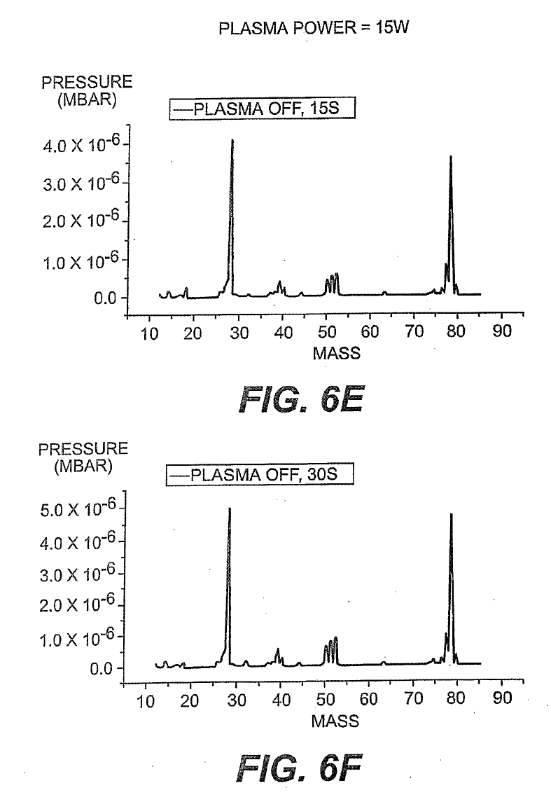

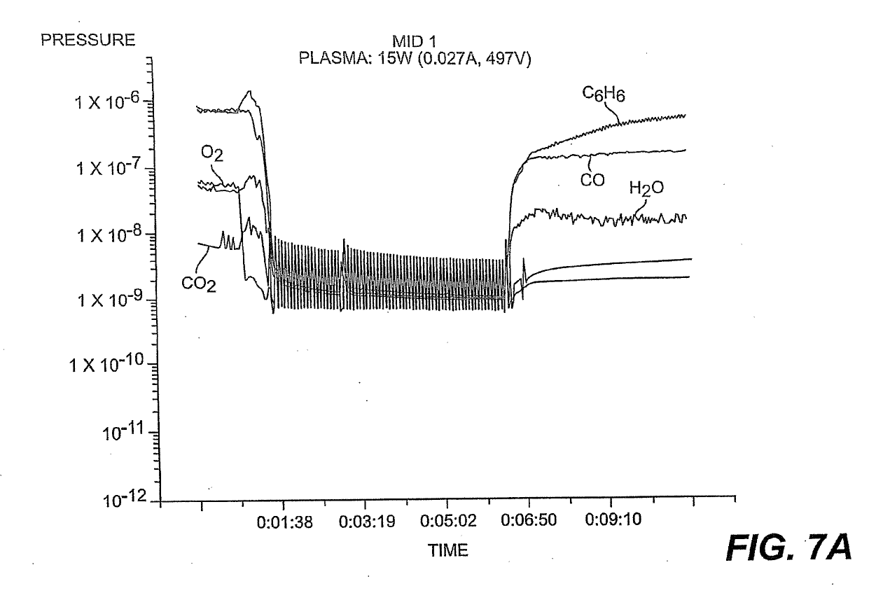

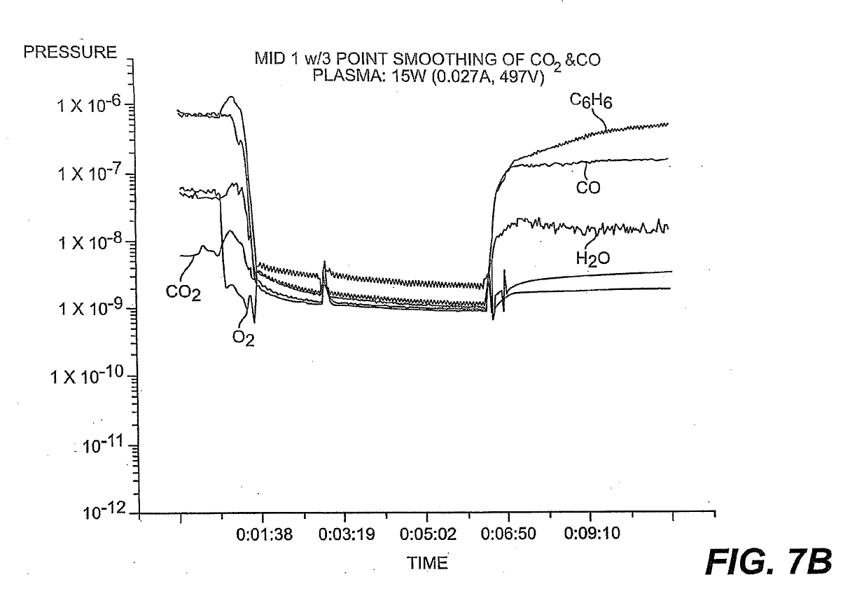

Once the plasma device was set up, the power supply 160a was turned on and benzene was allowed to flow from the reservoir 136 into the inner volume 207 of the HCS 112. The flow rate of the benzene was estimated by measuring the flow rate of argon required to maintain the same chamber pressure under similar conditions. QMS spectra and the chamber pressure were recorded as shown in the chromatographs of FIGS. 6A-6F and the multiple ion detection (MID) plots FIGS. 7A and 7B.

As shown in FIGS. 7A and 7B, benzene cracked to yield CO, O₂, CO₂ and H₂O when passed through the plasma. These byproducts of benzene cracking were trapped on the inner surface of the HCS 112, which resulted in the fall of the QMS signal and the chamber pressure, as shown in FIGS. 6A-6D. The chamber pressure continued to fall due to the cracking of benzene until the plasma became unstable and shut off (see FIGS. 6E-6F). The MID plots in FIGS. 7A and 7B present consolidated data showing the removal of benzene byproducts from the gas phase during operation of the system 400.

### Example 2

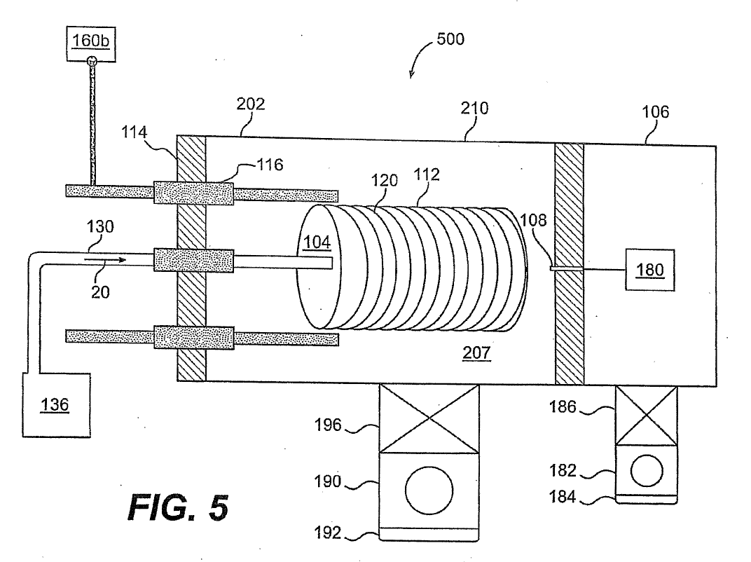

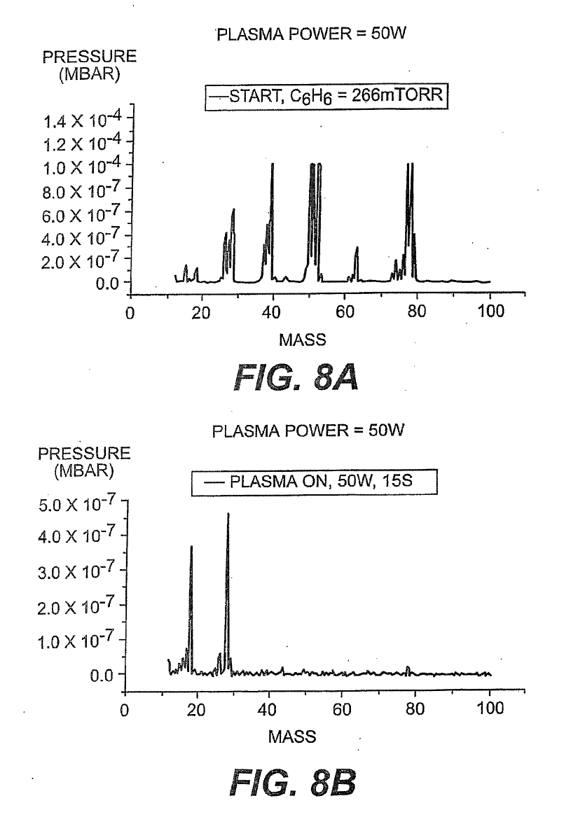

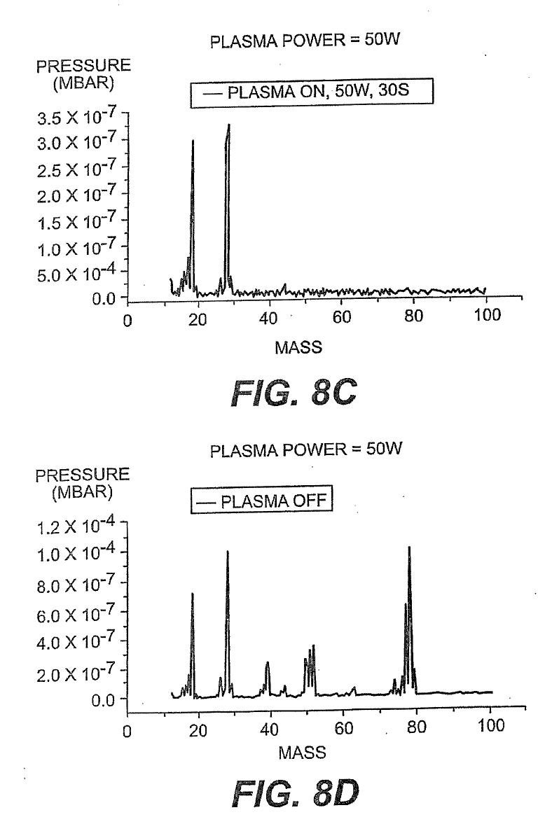

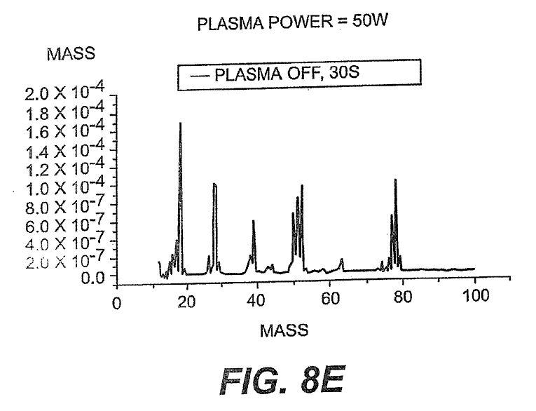

A hollow cathode source plasma system 500, shown in FIG. 5, was constructed and operated in similar fashion to the system 400 of Example 1, except that the device 210 was connected to a 50 watt power supply 160b. QMS spectra and the chamber pressure were recorded as shown in FIGS. 8A-8E under both “plasma on” and “plasma off conditions.”

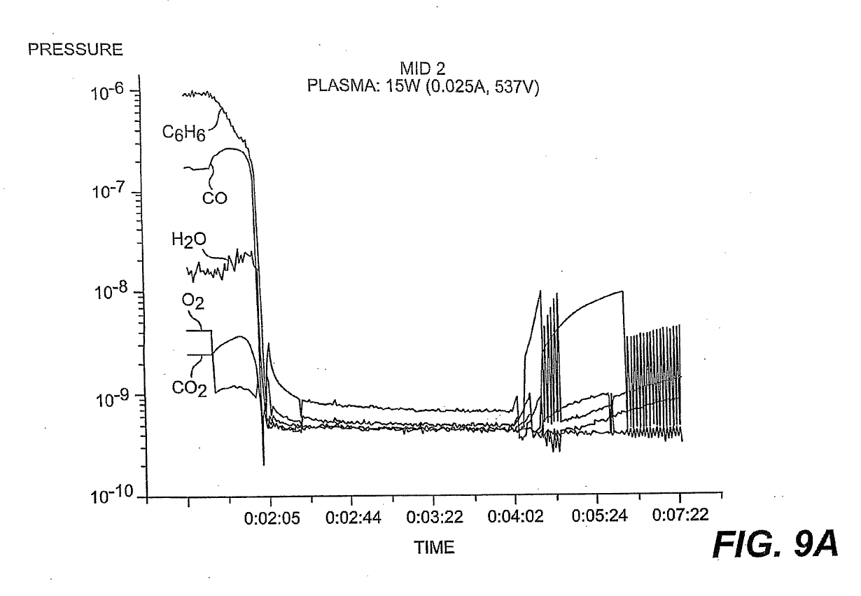

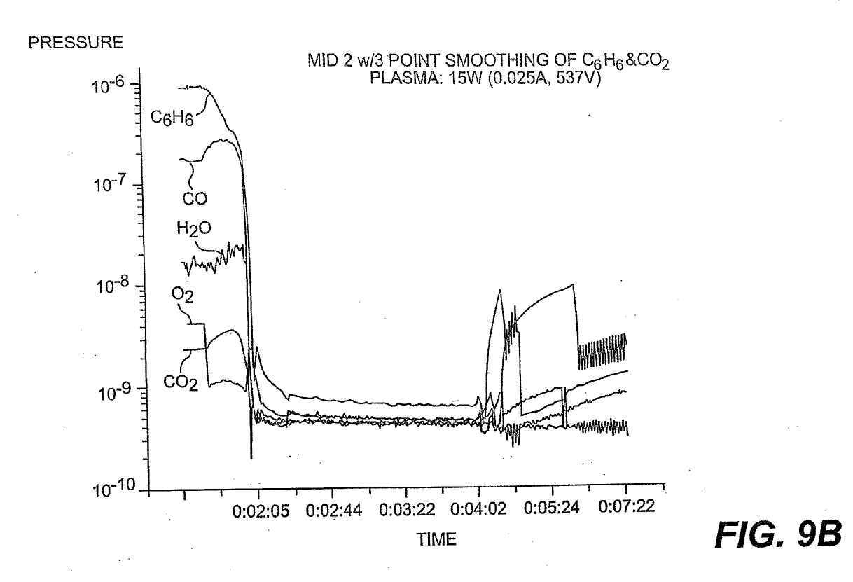

As shown in FIGS. 9A-9B, benzene cracked to yield CO, CO₂ and H₂O when passed through the plasma. As in the previous example, these byproducts of benzene cracking were trapped on the inner surface of the HCS 112, which lead to the fall of the QMS signal and the chamber pressure, as shown in FIGS. 8A-8C. The chamber pressure continued to fall due to the cracking of benzene until the plasma became unstable and shut off (see FIGS. 8D-8E). FIGS. 8A and 8B indicate the removal of benzene byproducts from the gas phase during operation of the system 500.

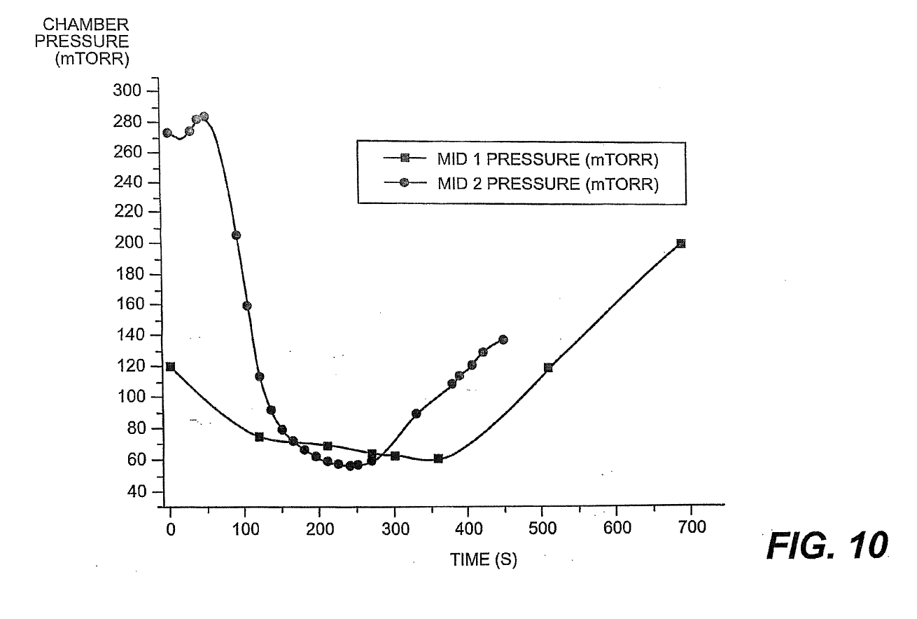

FIG. 10 provides comparative MID plots for experimental systems 300 and 400. Instead of partial pressure of the gases of interest shown in FIGS., 7A, 7B, 9A and 9B, FIG. 10 shows the total pressure of the cathode 112 in Examples 1 and 2.

In Examples 1 and 2, the successful cracking of benzene and trapping of benzene byproducts on the inner surface of the HCS demonstrate that HCS pollution abatement systems as disclosed herein can successfully eliminate chemical pollutants. The confined plasma in the hollow cathode is an energetic species. The break up of benzene shows that there is enough energy in the plasma to break the chemical bonds in benzene. Since the bonds in benzene are strong, the above examples illustrate that the disclosed HCS pollution abatement systems can successfully eliminate other chemical pollutants as well. Furthermore, microbiological species typically have less bonding energy than organic chemicals, and therefore the disclosed HCS pollution abatement systems are also capable of eliminating many microbiological organisms. Also, the confined plasma in a hollow cathode is even more energetic than the conventional plasma sources for decontaminating surfaces, and HCS systems will therefore also be effective in decontaminating surfaces.

The foregoing description illustrates and describes only selected embodiments, but it is to be understood that modifications within the scope of the inventive concept as expressed herein are possible, commensurate with the above teachings, and/or within the skill or knowledge of the relevant art. Accordingly, the description is not intended to limit the invention to the form disclosed herein. Also, it is intended that the appended claims be construed to include alternative embodiments, not explicitly defined in the detailed description.

We claim:

1. A pollution abatement system comprising:
a plasma generator, wherein said plasma generator comprises a hollow cathode source disposed in a housing and an anode spaced from said hollow cathode source;
a power supply connected to said plasma generator;
a feed gas supply comprising a feed gas suitable for generating plasma within an inner volume of the hollow cathode;
a feed gas tube extending from said feed gas supply into the inner volume of said hollow cathode source and arranged to carry a first gas stream comprising the feed gas;
a contaminated gas source comprising a contaminated gas;
a contaminated gas tube in communication with said contaminated gas source and connected to introduce a second gas stream comprising the contaminated gas to said first gas stream; and
a gas outlet in communication with an inner chamber of the housing and arranged to remove remediated gas from said plasma generator.

2. The pollution abatement system of claim 1, wherein the contaminated gas comprises one or more of the following agents: at least one chemical pollutant, at least one volatile organic compound and at least one microorganism.

3. The pollution abatement system of claim 1, wherein the pollution abatement system is an air cleaning system, wherein the contaminated gas source is a building or room and wherein the contaminated gas is air from the building or room.

4. The pollution abatement system of claim 1, wherein the pollution abatement system is a waste gas cleaning system for an industrial process, wherein the contaminated gas source is equipment used in the industrial process and wherein the contaminated gas is waste gas generated by the industrial process.

5. The pollution abatement system of claim 1, wherein the feed gas tube and the contaminated gas tube are separate gas tubes in selective communication with each other, and the contaminated gas is introduced from the contaminated gas tube into the feed gas tube as a second gas stream.

6. The pollution abatement system of claim 1, wherein the feed gas tube and the contaminated gas tube are the same gas tube, and the feed gas includes the contaminated gas.

7. The pollution abatement system of claim 6, wherein the pollution abatement system is an exhaust system for a motor vehicle, wherein the plasma generator is a catalytic converter, wherein the feed gas supply is an engine in the motor vehicle and wherein the feed gas is exhaust gas from the engine.

8. The pollution abatement system of claim 1, comprising:
a mass spectrometry chamber in communication with the inner volume of the hollow cathode through an orifice and containing a quantum mass spectrometer; and
a turbomolecular pump arranged to supply molecules to the quantum mass spectrometer for mass spectrometry.

9. A pollution abatement system comprising:
a plasma generator, wherein said plasma generator comprises a hollow cathode source disposed in a housing and an anode spaced from said hollow cathode source;
a power supply connected to said plasma generator;
a feed gas supply comprising a feed gas suitable for generating plasma within an inner volume of the hollow cathode;
a feed gas tube extending from said feed gas supply into the inner volume of said hollow cathode source and arranged to carry a first gas stream comprising the feed gas;
a gas outlet in communication with an inner chamber of the housing and arranged to remove gas from said plasma generator; and
a contaminated article disposed inside the hollow cathode source and arranged to be decontaminated by exposure to the plasma, wherein the contaminated article is contaminated by at least one of the following agents: a chemical pollutant, a volatile organic compound and a microorganism.

10. A method for removing contaminants from a gas, comprising:
providing a plasma generator, wherein said plasma generator comprises a hollow cathode source disposed in a housing and an anode spaced from said hollow cathode source;
powering said plasma generator with a power source;
supplying a feed gas to an inner volume of the hollow cathode so as to generate plasma within said inner volume;
supplying a contaminated gas to said inner volume and passing said contaminated gas through said plasma so as to break down said contaminated gas into remediated gas and byproducts; and
removing said remediated gas from said plasma generator.

11. The method of claim 10, comprising trapping said byproducts on an inner surface of the hollow cathode.

12. The method of claim 11, comprising allowing said byproducts to accumulate on said inner surface until a pressure within said inner volume falls to a level low enough to cease generation of said plasma.

13. The method of claim 12, comprising, subsequent to cessation of generation of said plasma, allowing said pressure within said inner chamber to rise to a level high enough to initiate plasma generation.

14. The method of claim 10, wherein said contaminated gas comprises an agent from the group consisting of: a chemical pollutant, a volatile organic compound and a microorganism.

15. The method of claim 10, wherein the feed gas and the contaminated gas are fed from separate sources through a respective feed gas tube and a contaminated gas tube which are in selective communication with respect to each other.

16. The method of claim 10, wherein the feed gas is supplied from a source containing the contaminated gas, and the feed gas and the contaminated gas are supplied to the inner volume of the hollow cathode from the source through a common gas tube.

17. A method for decontaminating an article, comprising:
providing a plasma generator, wherein said plasma generator comprises a hollow cathode source disposed in a housing and an anode spaced from said hollow cathode source;
placing the article inside the hollow cathode source, wherein a surface of the article is contaminated by at least one of the following agents: a chemical pollutant, a volatile organic compound and a microorganism;
powering said plasma generator with a power source;
supplying a feed gas to an inner volume of the hollow cathode so as to generate plasma within said inner volume;
exposing said surface of said article to said plasma so as to break down said agent into byproducts; and
removing uncontaminated gas from said plasma generator.

18. The method of claim 17, comprising trapping said byproducts on an inner surface of the hollow cathode.

19. The method of claim 18, comprising allowing said byproducts to accumulate on said inner surface until a pressure within said inner volume falls to a level low enough to cease generation of said plasma.

20. The method of claim 19, comprising, subsequent to cessation of generation of said plasma, allowing said pressure within said inner chamber to rise to a level high enough to initiate plasma generation.
